(19) United States
(12) Patent Application Publication
(10) Pub. No.: US 2023/0085426 A1
(43) Pub. Date: Mar. 16, 2023

(54) EFFICIENT AND INTELLIGENT SOURCE REDUCTIONS FOR QUERYING MULTIPLE SOURCES OVER AN EXTERNAL NETWORK

(71) Applicant: Surescripts, LLC, Arlington, VA (US)

(72) Inventors: Joseph Jerome Athman, Minneapolis, MN (US); Jeffrey Paul Larsen, Lakeville, MN (US); Michael James Androff, New Brighton, MN (US); Matthew Alen Bates, Woodbury, MN (US); Eric James Mulek, Golden Valley, MN (US); Kelly Frances Bundy, Shakopee, MN (US)

(21) Appl. No.: 17/476,721

(22) Filed: Sep. 16, 2021

(51) Int. Cl. G16H 10/60; G06F 16/9035; G06F 16/9038

(52) U.S. Cl. CPC G16H 10/60; G06F 16/9035; G06F 16/9038

(57) ABSTRACT

Systems, methods, and devices are described for efficient and intelligent source reductions for querying multiple sources over an external network. A medical history request for a patient is received from a requestor via a network. A set of providers is identified from a provider database based at least on the history request, and a set of physical addresses is generated based at least on the set of providers. The set of physical addresses are filtered according to filter criteria to generate a filtered set of physical addresses. Computing systems, of respective providers, which respectively correspond to the filtered set of physical addresses are queried over an external network to generate a query result that includes history information for the patient and that is representative of querying each computing system associated with the unfiltered set of physical addresses.

### BACKGROUND

Medical care providers generally inquire about a patient's medical history. Standard practice for locating medical records is for a provider to ask the patient where they have received care. Some current solutions involve systems that query providers identified by the patient as well as providers within a general geographic area surrounding a patient's home address and the requesting clinic's address, however, providers may be missed due to incompleteness or inaccuracy of the patient's memory or the geographic area. Furthermore, existing systems have inefficiencies in, or incapability for, querying enough computing systems associated with providers to determine the patient's medical history.

### BRIEF SUMMARY

Methods, processing systems, and apparatuses are described for efficient and intelligent source reductions for querying multiple sources over an external network, substantially as shown in and/or described herein in connection with at least one of the figures, as set forth more completely in the claims. A medical history request for a patient is received from a requestor via a network. A set of providers is identified from a provider database based at least on the history request, and a set of physical addresses is generated based at least on the set of providers. The set of physical addresses are filtered according to filter criteria to generate a filtered set of physical addresses that includes at least one less physical address than the set of physical addresses. Computing systems, of respective providers, respectively corresponding to ones of the filtered set of physical addresses are queried over the network to generate a query result that includes history information for the patient. The query result is representative of querying each computing system associated with the unfiltered set of physical addresses.

### BRIEF DESCRIPTION OF THE DRAWINGS/FIGURES

The accompanying drawings, which are incorporated herein and form a part of the specification, illustrate embodiments and, together with the description, further serve to explain the principles of the embodiments and to enable a person skilled in the pertinent art to make and use the embodiments.

FIG. 1 shows a block diagram of a computing system that includes a host for efficient and intelligent source reductions for querying multiple sources over an external network, according to an example embodiment.

FIG. 2 shows a block diagram of a network of computing systems that includes a host for efficient and intelligent source reductions for querying multiple sources over an external network, according to an example embodiment.

FIG. 3 shows a block diagram of a record locating and exchange system for efficient and intelligent source reductions for querying multiple sources over an external network, according to an example embodiment.

FIG. 4 shows a flowchart of a method for efficient and intelligent source reductions for querying multiple sources over an external network, according to an example embodiment.

FIG. 5 shows a block diagram of a provider identifier execution flow, according to an example embodiment.

FIG. 6 shows a block diagram of an address filter execution flow, according to an example embodiment.

FIG. 7 shows a flowchart of a method for efficient and intelligent source reductions for querying multiple sources over an external network, according to an example embodiment.

FIG. 8 shows a diagram of a region map for understanding locating and filtering techniques described herein.

FIG. 9 shows a flowchart of a method for efficient and intelligent source reductions for querying multiple sources over an external network, according to an example embodiment.

FIG. 10 shows a flowchart of a method for efficient and intelligent source reductions for querying multiple sources over an external network, according to an example embodiment.

FIG. 11 shows a diagram of regional density for understanding locating and filtering techniques described herein.

FIG. 12 shows a flowchart of a method for efficient and intelligent source reductions for querying multiple sources over an external network, according to an example embodiment.

FIG. 13 shows a flowchart of a method for efficient and intelligent source reductions for querying multiple sources over an external network, according to an example embodiment.

FIG. 14 shows a block diagram of a processing device/system in which the techniques disclosed herein may be performed and the embodiments herein may be utilized.

Embodiments will now be described with reference to the accompanying drawings. In the drawings, like reference numbers indicate identical or functionally similar elements. Additionally, the left-most digit(s) of a reference number identifies the drawing in which the reference number first appears.

### DETAILED DESCRIPTION

I. Introduction

The present specification discloses numerous example embodiments. The scope of the present patent application is not limited to the disclosed embodiments, but also encompasses combinations of the disclosed embodiments, as well as modifications to the disclosed embodiments.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

If the performance of an operation is described herein as being “based on” one or more factors, it is to be understood that the performance of the operation may be based solely on such factor(s) or may be based on such factor(s) along with one or more additional factors. Thus, as used herein, the term “based on” should be understood to be equivalent to the term “based at least on.”

In the discussion, unless otherwise stated, adjectives such as “substantially,” “approximately,” and “about” modifying a condition or relationship characteristic of a feature or features of an embodiment of the disclosure, are understood to mean that the condition or characteristic is defined to be within tolerances that are acceptable for operation of the embodiment for an application for which it is intended.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Still further, it should be noted that the drawings/figures are not drawn to scale unless otherwise noted herein.

Numerous exemplary embodiments are now described. Any section/subsection headings provided herein are not intended to be limiting. Embodiments are described throughout this document, and any type of embodiment may be included under any section/subsection. Furthermore, it is contemplated that the disclosed embodiments may be combined with each other in any manner. That is, the embodiments described herein are not mutually exclusive of each other and may be practiced and/or implemented alone, or in any combination.

II. Example Record Locating and Exchange System Embodiments

The example techniques and embodiments described herein provide for methods, systems, and apparatuses for efficient and intelligent source reductions for querying multiple sources over an external network, which may include locating and exchanging historic records based on machine-generated data resources and filter criteria. The example techniques and embodiments described herein may be adapted to various types of systems and devices, for example but without limitation, computing systems (e.g., computers/computing devices such as desktops, laptops, etc., and servers, enterprise computing systems, etc.), communication devices (e.g., cellular and smart phones, etc.), and/or the like, that communicate information and data that relates to, without limitation, patient demographics, medical history, provider data, etc., in different ways, in accordance with embodiments. For instance, computing systems that communicate over a network for locating and exchanging historical records of a patient may be configured according to the described embodiments and techniques for efficient and intelligent source reductions for querying multiple sources over an external network.

While the embodiments herein may be described with respect to various computing systems and implementations as conceptual and/or illustrative examples for descriptive consistency, other types of electronic and communication devices and implementations are also contemplated for implementing the disclosed techniques. It is contemplated herein that in various embodiments and with respect to the illustrated figures of this disclosure, one or more components described and/or shown may not be included and that additional components may be included.

In the context of locating and exchanging historic records of a patient, prior solutions rely on a patient to recite from memory previous medical history and providers they interacted with. The electronic systems of the recited list of providers may be queried for historic records of the patient. Exemplary embodiments herein, however, provide for the ability to identify additional providers based at least on one or more of transactional data, demographic data, geographic data, provider data, and/or the like. Transactional data may include, but is not limited to, medication prescriptions, past diagnosis codes, medical records, clinic visits, prescribing providers, prescription pickup locations, insurance information, and/or the like. Geographic data pertaining to the patient may include, but is not limited to, the patient's home address, the patient's work address, locations the patient has traveled to (e.g., recent vacations, work-related travel, addresses of relatives, etc.), past address information (e.g., former employer's address, past residential addresses), address type (e.g., single family home, multi-family home, apartment complex, dorm, etc.), and/or the like. Demographic data pertaining to the patient may include, but is not limited to, age, race, gender, marital status, income, education, employment, and/or the like. Provider data may include, but is not limited to, geographic data pertaining to the provider, demographic data pertaining to the provider, provider's recent activity (e.g., recent transactional data with the patient or another patient, opening a new office, closing an office, etc.), provider's responsiveness (e.g., accuracy of past query responses, accuracy of past record matches, turnaround time of past query responses, etc.), and/or the like.

The identified set of providers may be used to generate a set of physical addresses for each identified provider representing computing systems to query for historical records pertaining to the patient. For example, a provider may operate in multiple geographic locations. In one non-limiting example, a provider may be a doctor who has a primary care office at a first physical address, holds office hours at a hospital at a second physical address, and holds office hours at an outpatient clinic at a third physical address. In another non-limiting example, a provider is a pharmacy with multiple physical addresses across a city, state, country, and/or the like.

In some situations, the set of physical addresses generated may be very large. For example, thousands of addresses may be identified through locating techniques and embodiments described herein. It may be inefficient, or even impossible, for a host system to query every respective computing system corresponding to each respective physical address associated with each identified provider. For example, constraints or inefficiencies of the network, the host system, or the respective computing system may limit the query capabilities of the host system. Furthermore, a particular computing system associated with a respective physical address may not be relevant to the patient (e.g., a specialist clinic for a diagnosis not pertaining to the patient, a computing system of a provider located in a geographic region the patient has never been to, a computing system that has been inactive for a set period of time, a computing system newly activated after the patient last saw a provider, etc.). Exemplary embodiments may further filter the set of physical addresses using various filter criteria (e.g., patient demographics, provider interactions, provider activity, medication history, prior diagnosis codes, insurance information, geographical information, family relationships, prescription pickup locations, vendor responsiveness, etc.) to generate a filtered set of physical address. In embodiments, this filtered set of physical addresses may be a subset of the generated set of physical addresses that represents physical addresses of providers that the patient likely interacted with.

Computer/computing systems corresponding to the filtered set of physical addresses may then be queried for historic records of the patient. The query result may represent querying each computer/computing systems associated with the generated set of physical addresses of each identified provider. Accordingly, efficient and intelligent source reductions for querying multiple sources over an external network are achieved by the embodiments herein.

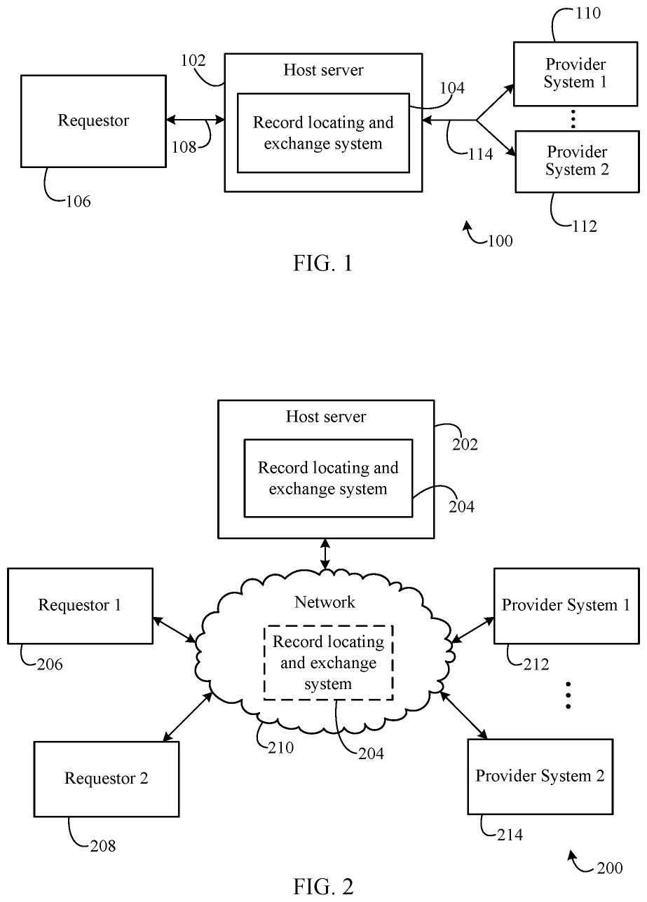

Systems and devices may be configured in various ways for efficient and intelligent source reductions for querying multiple sources over an external network. For instance, in FIG. 1, a block diagram of a network of computing systems 100 (“system 100” herein) that includes a record locating and exchange system 104 is shown, according to an example embodiment. System 100 includes a host server 102 that may include one or more processing devices such as, but not limited to, servers. Record locating and exchange system 104 is illustrated as a portion of host server 102. Additionally, system 100 includes a requestor system 106 that communicates electronically with host server 102 via a communication link 108, and a first provider system 110 and a second provider system 112 that communicate electronically with host server 102 via a communication link 114. Requestor system 106, first provider system 110, and second provider system 112 may also include one or more processing devices such as, but not limited to, servers and client devices such as laptop/desktop computers and computer terminals, personal handheld devices, etc.

Host server 102 may comprise one or more computers/servers of a host entity facilitating access to record locating and exchange system 104 by requestor system 106, first provider system 110, and/or second provider system 112, according to embodiments. Host server 102 may include geographically distributed computers/servers, a rack server system(s), a stand-alone server(s), cloud-based and/or on-premise servers, etc., in various embodiments. Example implementations of host server 102 also provide for host server 102 being a third-party entity with respect to requestor system 106, first provider system 110, and/or second provider system 112.

Requestor system 106, first provider system 110, and/or second provider system 112 may comprise one or more computers/servers of an entity, such as a vendor service(s), a doctor or doctor's office (including nurses and/or staff), a pharmacy, a patient, and/or the like as noted herein, that is associated with a history request. In other embodiments, requestor system 106, first provider system 110, and/or second provider system 112 are associated with users, purchasers or consumers, retailers, grocers, wholesalers, shipping/delivery entities, service providers, etc. It is also contemplated herein that requestor system 106 may be a provider system in some embodiments, and that first provider system 110, and/or second provider system 112 may be requestor systems in some embodiments.

Communication link 108 and/or communication link 114 may each comprise at least one network or direct communication connection, or any combination thereof, between host server 102 and requestor system 106, first provider system 110, and/or second provider system 112, that enables communication messages such as requests/responses for historic records, or other communication messages, as described herein, along with any associated electronic messages required. As used herein, the term “messages,” “communication messages,” etc., includes without limitation electronic communications data, information, packets, and/or the like, associated with history requests, responses thereto, etc. In embodiments, communication link 108 and/or communication link 114 may comprise wired and/or wireless portions of one or more networks such as networks of the host entity and requestors, including enterprise networks, the Internet, etc.

Record locating and exchange system 104 may comprise hardware and/or software components configured to perform operations for efficient and intelligent source reductions for querying multiple sources over an external network, as further described herein. As one example, requestor system 106 comprises a computing device of a doctor or doctor's office that the patient visits, and the doctor may request medical history information of the patient via their computing device from record locating and exchange system 104 via communication link 108, and upon receipt, record locating and exchange system 104 is configured to queue requests, according to embodiments. It may be subsequently determined by record locating and exchange system 104, either by patient data, provider data, and/or the like, as described herein, that first provider system 110 (and/or second provider system 112, in embodiments) may have historic records pertaining to the patient, and record locating and exchange system 104 is configured to query first provider system 110 via communication link 114 for historic data of the patient. Received data from providers, such as first provider system 110 in this scenario, may be aggregated and returned as a response to the requestor's request. Example implementations of record locating and exchange system 104 also provide for record locating and exchange system 104 being a third-party entity with respect to requestor system 106, first provider system 110, and/or second provider system 112.

In embodiments, one or more of any component shown in system 100 of FIG. 1 may be present in various implementations. Furthermore, while only first provider system 110 and second provider system 112 are shown in FIG. 1, the ellipsis between them indicates that system 100 may include many such provider systems.

Turning now to FIG. 2, a block diagram of a network of computing systems 200 is shown, according to an embodiment. Network of computing systems 200 (“system 200” herein) may be a further embodiment of system 100 of FIG. 1. System 200 includes a host server 202 having a record locating and exchange system 204 hosted/executed thereby, which may be embodiments of host server 102 and record locating and exchange system 104 of FIG. 1, respectively. System 200 also includes a first requestor system 206 and a second requestor system 208, which may be embodiments of requestor system 106 of FIG. 1, and a first provider system 212 and a second provider system 214, which may be embodiments of first provider system 110 and/or second provider system 112 of FIG. 1. The above-described components of system 200 may be communicatively coupled or link to each other via a network 210.

As noted, host server 202 may be a further embodiment of host server 102 of FIG. 1, and, for the purposes of illustration for FIG. 2, is configured the same, or substantially the same, as host server 102 above. Network 210 may be a further embodiment of communication link 108 and/or communication link 114 of FIG. 1. Network 210 may comprise at least one network and/or direct connection (i.e., a communication link), or any combination thereof. That is, network 210 may be any combination of the Internet, the “cloud”, direct communication links, business and/or enterprise networks, and/or the like.

That is, network 210 is configured to communicatively couple host server 202, first requestor system 206, second requestor system 208, first provider system 212, and second provider system 214 to each other. Accordingly, the network of computing systems shown as system 200 is configured as a further embodiment of the network of computing systems shown as system 100 of FIG. 1 in that the record locating and exchange system 204 of host server 202 is similarly configured to perform operations for efficient and intelligent source reductions for querying multiple sources over an external network. In some embodiments, such as cloud-based implementations, record locating and exchange system 204 may be implemented as a service or application via network 210.

With respect to FIG. 2, while shown for illustrative simplicity and brevity as including a single host (e.g., host server 202), two requestor systems, and two provider systems, it is contemplated herein that system 200 may include more or fewer of any of these components, as described herein or as otherwise would be understood by persons of skill in the relevant art(s) having the benefit of this disclosure, in embodiments. For instance, any number of requestor systems and/or provider systems may be supported for locating and exchanging historic records based on data resources and/or filter criteria, where the requestor systems are related to or associated with each other, or not, and where the provider systems are related to or associated with each other, or not. Additionally, first requestor system 206, second requestor system 208, first provider system 212, and/or second provider system 214 may be systems comprising computing devices for doctors or doctor's offices (including nurses and/or staff), patients (e.g., for historic record reference), consumers/buyers, vendor service(s), prescription fulfiller (e.g., pharmacies, retailers, grocers, shipping/delivery entities, service providers, etc.), etc., as described herein.

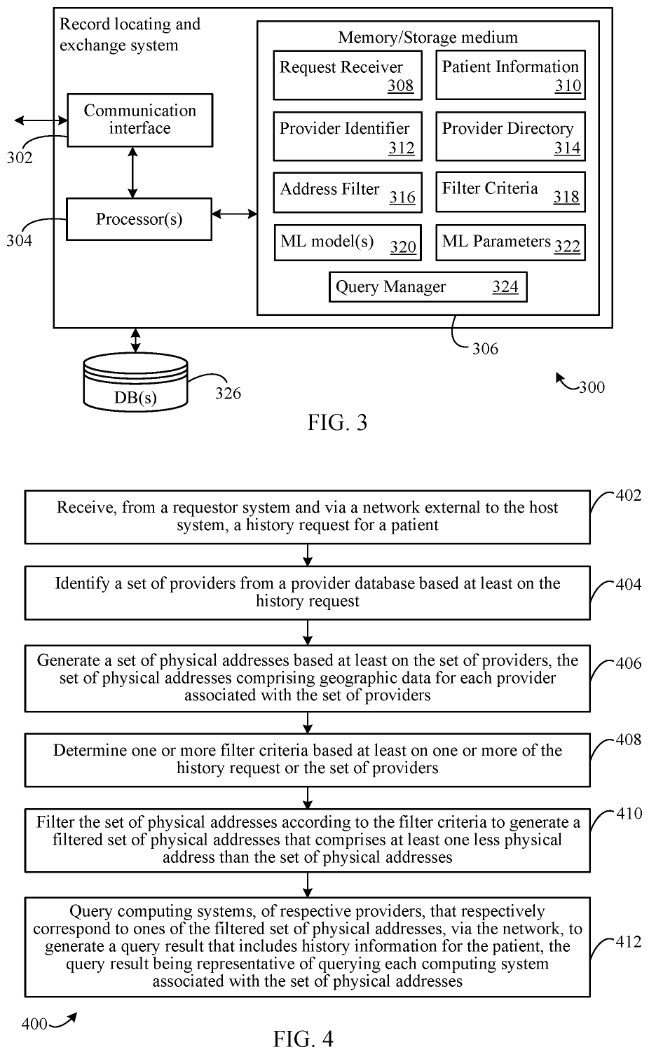

Turning now to FIG. 3, a block diagram of a system 300 is shown for efficient and intelligent source reductions for querying multiple sources over an external network, according to an example embodiment. System 300 as exemplarily illustrated and described is configured to be an embodiment of routing system 204 of system 200 in FIG. 2. System 300 is described as follows.

System 300 may comprise a computing device or system which is any type of server or computing systems, as mentioned elsewhere herein, or as otherwise known, including without limitation cloud-based systems, on-premises servers, distributed network architectures, and/or the like. As shown in FIG. 3, computing system 300 includes one or more processors (“processor”) 304, one or more of a memory and/or other physical storage device (“memory”) 306, as well as one or more communication interfaces 302 which may be network interfaces in embodiments.

System 300 is illustrated as including a request receiver 308, a provider identifier 312, an address filter 316, and a query manager 324 configured to perform operations for efficient and intelligent source reductions for querying multiple sources over an external network, according to embodiments.

Processor 304 and memory 306 may respectively be any type of processor circuit(s)/system(s) and memory that is described herein, and/or as would be understood by a person of skill in the relevant art(s) having the benefit of this disclosure. Processor 304 and memory 306 may each respectively comprise one or more processors or memories, different types of processors or memories (e.g., at least one cache for query processing), remote processors or memories, and/or distributed processors or memories. Processor 304 may be multi-core processors configured to execute more than one processing thread concurrently. Processor 304 may comprise circuitry that is configured to execute computer program instructions such as, but not limited to, embodiments of request receiver 308, provider identifier 312, address filter 316, and/or query manager 324, including one or more sub-components thereof, which may be implemented as computer program instructions, as described herein.

Memory 306 includes volatile storage portions such as a random access memory (RAM) and/or persistent storage portions such as hard drives, non-volatile RAM, and/or the like, in embodiments, to store or be configured to store computer program instructions/code for efficient and intelligent source reductions for querying multiple sources over an external network as described herein, as well as to store other information and data described in this disclosure including, without limitation, request receiver 308, provider identifier 312, address filter 316, query manager 324, and/or the like, in different embodiments. Memory 306 also includes storage of data, information, and/or machine learning (ML) models, according to embodiments. For instance, patient information 310, provider directory 314, filter criteria 318, ML model(s) 320, and/or ML parameters 322 may be stored in memory 306 according to embodiments. Such information and data may be stored in various forms, such as in database(s), lists, unstructured data storage, and/or the like.

Communication interface 302 may be any type or number of wired and/or wireless communication or network adapters, modems, etc., configured to enable system 300, to communicate intra-system with components thereof, as well as with other devices and/or systems over a network, such as communications between system 300 and other devices, systems, hosts, as described for system 100 in FIG. 1 and/or system 200 in FIG. 2, over a network such as network 210.

System 300 may also include one or more databases (DBs) 326, in embodiments. DB(s) 326 may include patient information 310, provider directory 314, filter criteria 318, local-host versions of compendia, and/or any other database/records information described herein. In embodiments, DB(s) 326 may be internal or external to host system 300, and in some embodiments, may comprise a portion of memory 306.

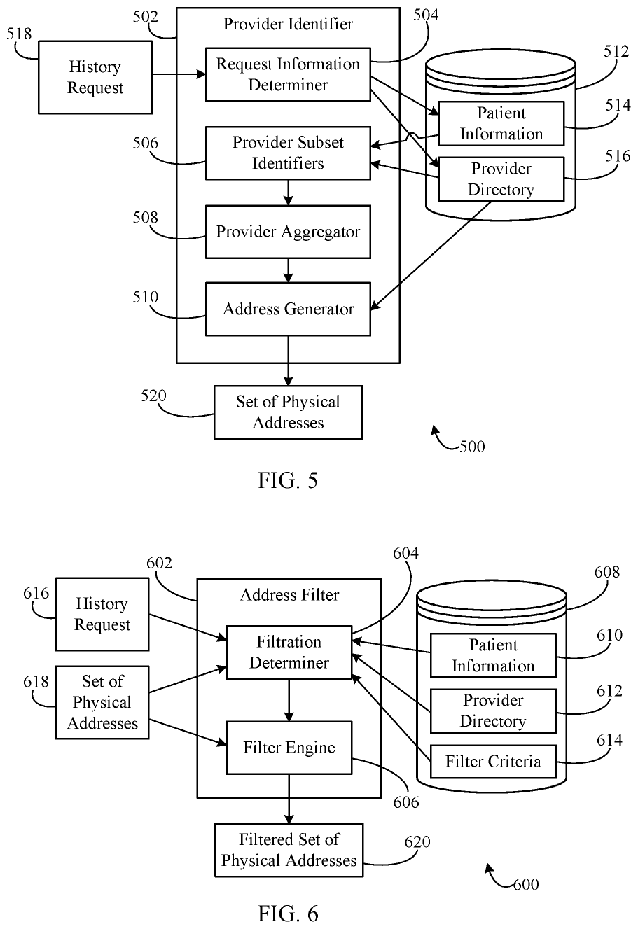

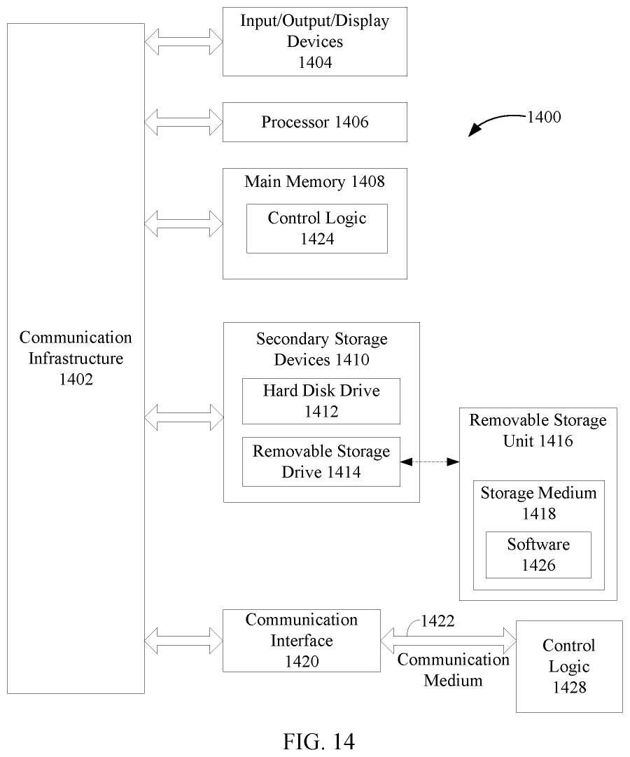

System 300 also includes additional components (not shown for brevity and illustrative clarity) including, but not limited to, components and subcomponents of other devices and/or systems herein, as well as those described below with respect to FIGS. 5-6 and/or FIG. 14, including software such as an operating system (OS), according to embodiments.

As noted above, system 300 may include request receiver 308, provider identifier 312, address filter 316, and/or query manager 324, which may be services or applications, e.g., embodied as software that executes on system 300, in embodiments. Request receiver 308 is configured to receive a history request from requestors or requestor systems, e.g., first requestor system 206 and/or second requestor system 208 in FIG. 2, as described herein. History requests from requestors may be queued in memory 306 by request receiver 308. Request receiver 308 may store all or a portion of data within the history request (e.g., patient identification, patient information, medication information, requestor information, etc.) in any appropriate data structure as patient information 310 (e.g., patient identification transactional data, patient demographics, provider interactions, medication history, prior diagnosis codes, insurance information, geographical information, family relationships, prescription pickup locations, etc.).

Provider identifier 312 is configured to identify a set of providers (e.g., doctor(s), physicians, clinics, telemedicine services, prescription fulfillers, etc.) from provider directory 314 based at least on data within the history request. Provider identifier 312 may also be configured to identify a set of additional providers via the data stored as patient information 310, data stored in provider directory 314, ML model(s) 320, ML parameters 322, and/or the like. In the context of identifying additional providers, provider identifier 312 may aggregate the set of providers identified based at least on data within the history request and the set of additional providers. Provider directory 314 may be any appropriate data structure that includes information associated with providers within the directory (e.g., provider identification, provider type, provider activity, physical addresses, practice area, responsiveness, etc.). Provider identifier 312 may also be configured to generate a set of physical addresses comprising geographic data for each provider associated with the set of providers.

Address filter 316 is configured to filter the set of physical addresses generated by provider identifier 312 according to filter criteria 318 (e.g., patient demographics, provider interactions, provider activity, medication history, prior diagnosis codes, insurance information, geographic information, family relationships, prescription pickup locations, vendor responsiveness, etc.) to generate a filtered set of physical addresses. In a non-limiting example, the filtered set of physical addresses comprises at least one less physical address than the set of physical addresses. That is, efficient and intelligent source reductions for querying multiple sources over an external network includes reducing candidate physical addresses, e.g., via filtering, to generate a manageable set of physical addresses to be queried, which prevents system crashes, reduces memory footprint, reduces processing cycles requires, and reduces network bandwidth, while at the same time, ensuring that physical addresses in the reduced, filtered set are determined as candidates from which the request can be satisfied.

Query manager 324 is configured to query computing systems corresponding to the filtered set of physical addresses, e.g., first provider system 212 and/or second provider system 214 of FIG. 2. Queries performed by query manager 324 are performed over a network, e.g., network 210 in FIG. 2, which may be an external network with respect to system 300, and which may include querying hundreds, thousands, or more computing systems for the filtered set of physical addresses in different scenarios and geographic regions, again ensuring that physical addresses in the reduced, filtered set are determined as candidates from which the request can be satisfied, but still without querying each computing system at each determined physical address.

As noted above, system 300 may include ML model(s) 320 and ML parameters 322. ML model(s) 320 may include a trained matching ML model that is trained on prior history requests and/or transactional data with ML parameters 322 directed to patient information (e.g., patient identification, transactional data, patient demographics, provider interactions, medication history, prior diagnosis codes, insurance information, geographical information, family relationships, prescription pickup locations, etc.). The trained matching ML model may match a patient associated with a history request to one or more other patients stored within a database, such as DB(s) 326. Information associated with the matched one or more other patients (e.g., transactional data, provider interactions, prescription pickup locations, etc.) may be used by provider identifier 312 to identify the set of providers and/or set of physical addresses. In embodiments, the trained matching ML model herein may be used to identify additional providers that may have interactions with the patient associated with a history request by matching a patient profile of the patient with information associated with profiles of other patients. In embodiments, scores/indices may be required to meet or exceed a threshold value to determine a positive match, and it is contemplated herein that matching scores/indices within a pre-determined amount of meeting/exceeding such a threshold may be identified for further refinement by the ML models herein. Accordingly, efficient and intelligent source reductions for querying multiple sources over an external network is further achieved based on the described embodiments.

Turning now to FIG. 4, a flowchart 400 is shown for efficient and intelligent source reductions for querying multiple sources over an external network, in accordance with an example embodiment. Further structural and operational examples will be apparent to persons skilled in the relevant art(s) based on the following descriptions. Flowchart 400 is described as follows with respect to system 200 of FIG. 2 and system 300 of FIG. 3.

Flowchart 400 begins at step 402 where a history request for a patient is received from a requestor system via a network external to a host. For example, a history request from first requestor system 206 or second requestor system 208 described for FIG. 2 is received via communication interface 302 of system 300 in FIG. 3. In embodiments, request receiver 308 may comprise program code that is executable/executing by/on processor(s) 304 of system 300 in FIG. 3 to store the history request as patient information 310 and/or provide the history request to provider identifier 312. In a non-limiting example, the history request is a medical history request for a patient from a physician or physician's office over network 210. This request is associated with historic medical records stored by a computing system associated with one or more providers, such as first provider system 212 and/or second provider system 214, e.g., as determined according to the steps of flowchart 400 described here. In embodiments, the history request may contain information pertaining to the patient (e.g., identification information, transactional data, patient demographics, prescription information, diagnosis codes, insurance information, geographical information, etc.) and/or information pertaining to the requestor (e.g., identification information, requestor type, transactional data, geographical information, requestor interactions, practice area, etc.)

In step 404, a set of providers is identified from a provider database based at least on the history request. For example, provider identifier 312 of system 300 in FIG. 3 is configured to identify a set of providers from provider directory 314 based at least on information in the history request. The set of providers may include information associated with the providers stored within the directory (e.g., provider identification, provider type, provider activity, physical addresses, practice area, responsiveness, etc.). In some embodiments, provider identifier 312 may update provider directory 314 with information in the history request pertaining to the set of providers.

In step 406, a set of physical addresses is generated based at least on the set of providers. For example, provider identifier 312 of system 300 in FIG. 3 is configured to identify a set of physical addresses for each provider associated with the set of providers. In embodiments, the set of physical addresses includes geographic data for each provider associated with the set of providers.

In step 408, filter criteria is determined based at least on one or more of the history request or the set of providers. For example, filter criteria 318 is selected by address filter 316 in FIG. 3 based at least on information in the history request and/or information in provider directory 314 associated with the set of providers. In embodiments, filter criteria 318 includes metrics to filter the set of physical addresses (e.g., patient demographics, provider interactions, provider activity, medication history, prior diagnosis codes, insurance information, geographic information, family relationships, prescription pickup locations, vendor responsiveness, etc.).

In step 410, the set of physical addresses are filtered according to the filter criteria to generate a filtered set of physical addresses. For example, address filter 316 of system 300 in FIG. 3 filters the set of physical addresses according to filter criteria 318. In embodiments, the filtered set of physical addresses comprises at least one less physical address than the set of physical addresses. That is, efficient and intelligent source reductions for querying multiple sources over an external network includes reducing candidate physical addresses, e.g., via filtering, to generate a manageable set of physical addresses to be queried, which prevents system crashes, reduces memory footprint, reduces processing cycles requires, and reduces network bandwidth, while at the same time, ensuring that physical addresses in the reduced, filtered set are determined as candidates from which the request can be satisfied.

In step 412, computing systems of respective providers corresponding to the filtered set of physical addresses are queried via the network to generate a query result including history information for the patient. For example, query manager 324 of system 300 in FIG. 3 sends queries requesting history information for the patient to first provider system 212 and/or second provider system 214 described for FIG. 2. In embodiments, first provider system 212 and second provider system 214 respectively correspond to ones of the set of physical addresses generated in step 406. First provider system 212 and second provider system 214 may respectively correspond to the same provider with different physical addresses, different providers with the same physical address, or different providers with different physical addresses. In a non-limiting example, first provider system 212 and/or second provider system 214 respectively correspond to a physical address that is filtered out of the set of physical addresses by the filtering in step 410 and are not subsequently queried in step 412. In embodiments, the query result is representative of querying each computing system associated with the set of physical addresses. For example, the network, e.g., network 210 in FIG. 2, which may be an external network with respect to system 300, may include hundreds, thousands, or more computing systems. In this context, embodiments described herein efficiently and intelligently ensure that the reduced, filtered set are determined as candidates from which the request can be satisfied, but still without querying each computing system at each determined physical address. Query results may then be provided to the requestor to satisfy their request received in step 402.

In the context of FIGS. 2-4, FIG. 5 will now be described. In FIG. 5, a provider identifier execution flow 500 (“execution flow 500”) is shown, according to an example embodiment. As illustrated in execution flow 500, a history request 518 is provided to provider identifier 502. History request 518 may be received by communication interface 302 of system 300 in FIG. 3 via network 210 of FIG. 2. Provider identifier 502 may be a further embodiment of provider identifier 312 of system 300. Provider identifier 502 may store or retrieve information from memory 512. Memory 512 may be a further embodiment of a portion of memory 306 of system 300. Memory 512 may include patient information 514 and provider directory 516, which respectively may be further embodiments of patient information 310 and provider directory 314 of system 300. Provider identifier 502 provides a set of physical addresses 520 as an output.

Provider identifier 502 may be a service or application, e.g., embodied as software that executes on system 300 of FIG. 3, in embodiments. In embodiments, provider identifier 502 is configured to perform functions including, but not limited to, step 404 and step 406 described with respect to FIG. 4. Provider identifier 502 may include request information determiner 504, provider subset identifiers 506, provider aggregator 508, and/or address generator 510, which may be sub-services, submodules, or sub-applications of provider identifier 502.

Request information determiner 504 is configured to determine information about the patient and/or requestor associated with history request 518. For example, request information determiner 504 may determine information about the patient and store the information as patient information 514. Request information determiner 504 may determine information about the requestor, for example the respective requestor(s) associated with first requestor system 206 and/or second requestor system 208 of FIG. 2, and store the information in provider directory 516.

Provider subset identifiers 506 may include one or more identifiers configured to identify one or more subsets of providers based at least on information from the history request. For example, provider subset identifiers 506 may identify a subset of providers based at least on one or more of history request 518, patient information 514, and/or provider directory 516. In a non-limiting example, a first provider subset identifier identifies a first subset of providers from provider directory 516 based on history request 518 and a second provider subset identifier identifies a second subset of providers from provider directory 516 based on patient information 514.

Provider aggregator 508 is configured to aggregate identified subsets of providers as generate a set of providers. For example, provider aggregator 508 aggregates identified subsets of providers identified by provider subset identifiers 506 as the set of providers. In the context of the non-limiting example described above with respect to provider subset identifiers 506, provider aggregator 508 may aggregate the first subset of providers and the second subset of providers as the set of providers.

In embodiments, provider subset identifiers 506 and provider aggregator 508 are configured to identify a set of providers from a provider database (e.g., provider directory 516) based at least on history request 518, such as described in step 404 in FIG. 4. In some embodiments, provider subset identifiers 506 may be a single identifier service or application that identifies the set of providers, and in such a case, provider aggregator 508 may be removed from provider identifier 502.

Address generator 510 is configured to generate a set of physical addresses based at least on the set of providers, as described in step 406 in FIG. 4. For example, address generator 510 generates set of physical addresses 520 comprising geographic data for each provider associated with the set of providers identified by provider subset identifiers 506 and/or provider aggregator 508. In embodiments, the set of providers may already contain information including set of physical addresses 520, or address generator 510 may cross-reference the set of providers with geographic data stored in provider directory 516 to generate set of physical addresses 520.

Provider identifier 502 may also include additional subcomponents (not shown for brevity and illustrative clarity). In some embodiments, one or more components of system 300 in FIG. 3 may be combined with provider identifier 502 as a single component or subcomponent. In some embodiments, one or more subcomponents of provider identifier 502 may be shared with, moved to, or part of another component of system 300 in FIG. 3. For example, request information determiner 504 may be included in request receiver 308 of system 300.

In the context of FIGS. 3-5, FIG. 6 will now be described. In FIG. 6, an address filter execution flow 600 (“execution flow 600”) is shown, according to an example embodiment. As illustrated in execution flow 600, a history request 616 and/or a set of physical addresses 618 are provided to an address filter 602. History request 616 may correspond to history request 518 of execution flow 500. Set of physical addresses 618 may correspond to set of physical addresses 520 generated by provider identifier 502 of FIG. 5. Address filter 602 may be a further embodiment of address filter 316 of system 300 in FIG. 3. In embodiments, address filter 602 may retrieve information from a memory 608. Memory 608 may be a further embodiment of a portion of memory 306 of system 300. Memory 608 may include patient information 610, a provider directory 612, and/or filter criteria 614, which respectively may be further embodiments of patient information 310, provider directory 314, and filter criteria 318 of system 300. Address filter 602 provides a filtered set of physical addresses 620 as an output.

Address filter 602 may be a service or application, e.g., embodied as software that executes on system 300 of FIG. 3, in embodiments. In embodiments, address filter 602 is configured to perform functions of including, but not limited to, step 408 and step 410 described with respect to FIG. 4. Address filter 602 may include a filtration determiner 604 and/or a filter engine 606, which may be sub-services, submodules, or sub-applications of address filter 602.

Filtration determiner 604 is configured to determine one or more filter criteria based at least on one or more of the history request or the set of providers, as described with respect to step 408 of FIG. 4. For example, filtration determiner 604 receives history request 616 and/or set of physical addresses 618 and determines one or more filter criteria from filter criteria 614. In some embodiments, filtration determiner 604 may retrieve information from patient information 610 and/or provider directory 612 to determine one or more filter criteria from filter criteria 614. In some embodiments, filter criteria may be predetermined for functions of address filter 602 by a component or subcomponent of system 300, an external database such as DB(s) 326 of FIG. 3, a user via a user interface of system 300, and/or the like.

Filter engine 606 is configured to filter the set of physical addresses according to the filter criteria to generate a filtered set of physical addresses, as described with respect to step 410 of FIG. 4. For example, filter engine 606 receives set of physical addresses 618 from provider identifier 502 (e.g., corresponding to set of physical addresses 520) and applies selected filter criteria from filtration determiner 604 to generate filtered set of physical addresses 620. In embodiments, filtered set of physical addresses 620 comprises at least one less physical address than set of physical addresses 618. That is, efficient and intelligent source reductions for querying multiple sources over an external network includes reducing candidate physical addresses, e.g., via the operation of address filter 602, to generate a manageable set of physical addresses to be queried, which prevents system crashes, reduces memory footprint, reduces processing cycles requires, and reduces network bandwidth, while at the same time, ensuring that physical addresses in the reduced, filtered set are determined as candidates from which the request can be satisfied.

Address filter 602 may also include additional subcomponents (not shown for brevity and illustrative clarity). In some embodiments, one or more components of system 300 in FIG. 3 may be combined with address filter 602 as a single component or subcomponent. In some embodiments, one or more subcomponents of address filter 602 may be shared with, moved to, or part of another component of system 300 in FIG. 3.

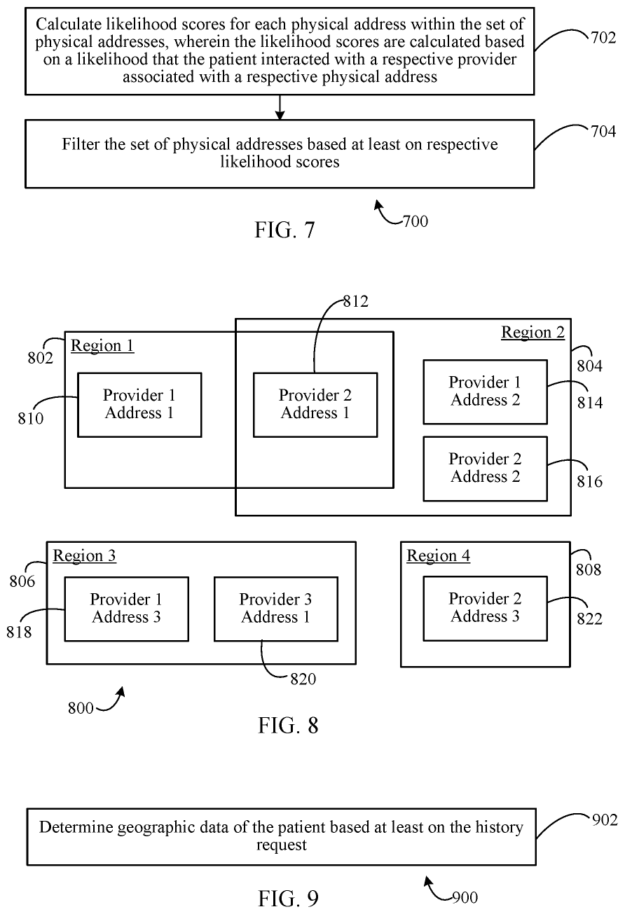

Turning now to FIG. 7, a flowchart 700 for efficient and intelligent source reductions for querying multiple sources over an external network, according to an example embodiment. Further structural and operational examples will be apparent to persons skilled in the relevant art(s) based on the following descriptions. Flowchart 700 may be an embodiment of flowchart 400 in FIG. 4, and is described as follows with respect to FIGS. 2-4 and 6. In embodiments, one or more steps of flowchart 700 may not be performed.

Flowchart 700 begins at step 702, which may be performed as a subset of steps 408 and/or 410 of flowchart 400 in FIG. 4. In step 702, likelihood scores are calculated for each physical address within the set of physical addresses. In this example embodiment, likelihood scores are calculated based on a likelihood that the patient interacted with a respective provider associated with a respective physical address. For example, filtration determiner 604 and/or filter engine 606 of address filter 602 in FIG. 6 calculates a likelihood that the patient associated with history request 616 has interacted with a respective provider associated with one or more of the addresses within set of physical addresses 618, e.g., based on at least one or more of information within history request 616, patient information 610, provider directory 612, and/or filter criteria 614. In a non-limiting example, a history request is made for a patient with a previous cancer diagnosis, and set of physical addresses 618 includes a first physical address of a primary care physician a patient has visited in the past year, a second physical address of an oncology center near the patient's home address, and a third physical address of a dialysis clinic associated with the patient's primary care provider. In this context, filtration determiner 604 calculates a likelihood scores based at least on patient information 610 and physical address type from filter criteria 614. That is, filtration determiner 604 calculates a relatively high likelihood score for the first physical address, a moderate likelihood score for the second physical address, and a relatively low likelihood score for the third physical address.

Flowchart 700 continues to step 704, which may be performed as a subset of step 410 of flowchart 400 in FIG. 4. In step 704, the set of physical addresses are filtered based at least on respective likelihood scores. For example, filter engine 606 of address filter 602 in FIG. 6 filters set of physical addresses 618 based at least on respective likelihood scores to generate filtered set of physical addresses 620. In some embodiments, filter engine 606 may remove physical addresses with a likelihood score at and/or lower than a set threshold, and/or the like (i.e., embodiments contemplate threshold comparisons for less than, less than or equal to, equal to, greater than or equal to, and greater than in different implementations). In this context, the set threshold may be pre-determined or variable. In the context of the non-limiting example described with respect to step 702, filter engine 606 may determine the third physical address is unlikely to have medical records pertaining to the patient and remove the third physical address to generate the filtered set of physical addresses 620.

In the context of set variable thresholds mentioned above, embodiments contemplate thresholds that vary according to factors such as, but not limited to, network bandwidth, an amount of regions identified, population densities of respective regions, clinic densities of respective regions, an amount of providers identified, an amount of physical addresses identified, responsiveness of respective providers, and/or the like. Furthermore, embodiments contemplate variable thresholds that are percentages of and/or ratios of one or more factors (e.g., percentage of network bandwidth available, ratio of clinics to residents of a region, percentage of providers active in the last month, average accuracy of provider responses, etc.). In a non-limiting example, the set threshold may change according to bandwidth of network 210 of FIG. 2. In this example, millions of history requests are made in a single day in network of computing systems 200, which includes host system 202 with a record locating and exchange system 204 and tens of thousands of computing systems associated with respective requestors (e.g., first requestor system 206 and/or second requestor system 208) and/or providers (e.g., first provider system 212 and/or second provider system 214). To reduce the load on network 210, filter engine 606 limits an address count of each filtered set of physical addresses 620 to the twenty respective physical addresses with the highest respective likelihood scores. While the address count is limited to twenty with respect to the non-limiting example above, it is contemplated herein that address count may be limited to an amount based on one or more of a set number, a percentage of the network bandwidth, a ratio of one or more factors, a percentage of one or more factors, and/or the like.

In the context of the method described in flowchart 700, different filter criteria of providers and/or physical addresses may be given different weight values when determining likelihood scores. For example, filtration determiner 604 of address filter 602 in FIG. 6 determines a first filter criteria contributes to a likelihood score more than a second filter criteria. In this context, filtration determiner 604 assigns a higher weight value to the first filter criteria and a lower weight value to the second filter criteria. In one non-limiting example, a provider's activity at a physical address may be given a higher weight value than the provider's responsiveness. In this context, filtration determiner 604 assigns a likelihood score to a first physical address of a first provider who is considered responsive but has not operated at the first physical address in the past year that is lower than a likelihood score assigned to a second physical address of a second provider who is considered less responsive but has routinely operated at the second physical address. As described above, filter engine 606 may remove physical addresses with a likelihood score at and/or lower than a set threshold, and/or the like (i.e., embodiments contemplate threshold comparisons for less than, less than or equal to, equal to, greater than or equal to, and greater than in different implementations).

In the context of the method described in flowchart 700, some embodiments described herein efficiently and intelligently perform source reductions based at least on respective likelihood scores of physical addresses. In this context, likelihood scores are representative of the likelihood that a patient interacted with a respective provider at a respective physical address. This method may be used to generate a manageable set of physical addresses to be queried, which prevents system crashes, reduces memory footprint, reduces processing cycle requires, and reduces network bandwidth, while at the same time, ensuring that physical addresses in the reduced, filtered set are determined as candidates from which the request can be satisfied.

III. Example Geographic Locating and Filtering System Embodiments

As described above, systems may be configured in various ways to perform their described functions. For instance, systems may be configured to identify and/or filter sets of providers and/or sets of physical addresses according to geographic data of a patient, of a provider, of a requestor, and/or the like, according to some embodiments. For example, geographic data may include, but is not limited to, the patient's home address, the patient's work address, physical addresses associated with one or more providers, physical addresses associated with one or more requestors, travel records of the patient, physical addresses associated with the patient's family members, etc.

FIG. 8 shows a diagram of a region map 800 (“map 800” herein) for understanding record locating and filtering techniques described herein. Map 800 as exemplarily illustrated and described may be used to understand functions of a record locating and exchange system for efficient and intelligent source reductions for querying multiple sources over an external network, such as system 300 of FIG. 3.

Map 800 includes a first region 802, a second region 804, a third region 806, and a fourth region 808, collectively referred to as “regions 802-808” herein. Each of these regions represents a geographic region covering a particular area. For example, a region may be one or more of a city, neighborhood, county, state, city-block, zip code, and/or the like. Physical addresses associated with one or more of patients, providers, and/or requestors may be located within one or more of regions 802-808. For example, as illustrated, map 800 shows several physical addresses associated with respective providers. First region 802 may include a first physical address of a first provider 810 and a first physical address of a second provider 812. Second region 804 may include first physical address of the second provider 812, a second physical address of the first provider 814, and a second physical address of the second provider 816. Third region 806 may include a third physical address of the first provider 818 and a first physical address of a third provider 820. Fourth region 808 may include a third physical address of the second provider 822. Collectively, the respective physical addresses are referred to as “physical addresses 810-822” herein.

As illustrated, map 800, regions 802-808 and physical addresses 810-822 in FIG. 8 are not drawn to scale. For example, regions may be a variety of different shapes and/or sizes. In some embodiments, each region within a map may be set to the same shape and/or size by default. Regions may be completely separate from one another, may overlap with one another, be separated by large distances (e.g., located within separate countries, states, counties, zip codes, cities, etc.), and/or the like. In some embodiments, physical addresses may overlap with one another. For example, physical addresses of respective providers (e.g. second physical address of the first provider 814 and second physical address of the second provider 816) may be geographically located within the same office building, hospital, medical complex, and/or the like. In embodiments, a map used for record locating and exchanging functions/operations may include many regions and each region may include many respective physical addresses. For example, a region or group of regions may include ones, tens, hundreds, thousands, or more computing systems associated with respective physical addresses.

Physical addresses may correspond to a computing system associated with a respective provider. Computing systems corresponding to physical addresses associated with a single provider (e.g., first physical address of the first provider 810, second physical address of the first provider 814, and third physical address of the first provider 818) may operate and/or store data independently, via an intra-network, or via a network. Computing systems corresponding to physical addresses associated with a single provider may be operated by different governing entities (e.g. a hospital, doctor's office, clinic, vendor, etc.). In a non-limiting example, first physical address of the first provider 810 may be a hospital associated with multiple providers and second physical address of the first provider 814 may be a doctor's office associated with the first provider.

Turning now to FIG. 9, a flowchart 900 for efficient and intelligent source reductions for querying multiple sources over an external network is shown, according to an example embodiment. Further structural and operational examples will be apparent to persons skilled in the relevant art(s) based on the following descriptions. Flowchart 900 may be an embodiment of flowchart 400 of FIG. 4, and is described as follows with respect to FIGS. 4 and 5. In embodiments, the step of flowchart 900 may not be performed.

Flowchart 900 includes step 902, which may be performed subsequent to step 402 or as a subset of step 404 of flowchart 400. In step 902, geographic data of a patient is determined based at least on a history request. For example, request information determiner 504 of provider identifier 502 in FIG. 5 determines geographic data of the patient based at least on information within history request 518. In embodiments, request information determiner 504 may store the geographic data of the patient as a portion of patient information 514. In some embodiments, request information determiner 504 may verify, amend, and/or append the determined geographic data of the patient to existing data stored within patient information 514. In a non-limiting example, the geographic data of the patient determined in step 902 may indicate anew home address associated with the patient. In this context, existing data stored within patient information 514 is expanded upon.

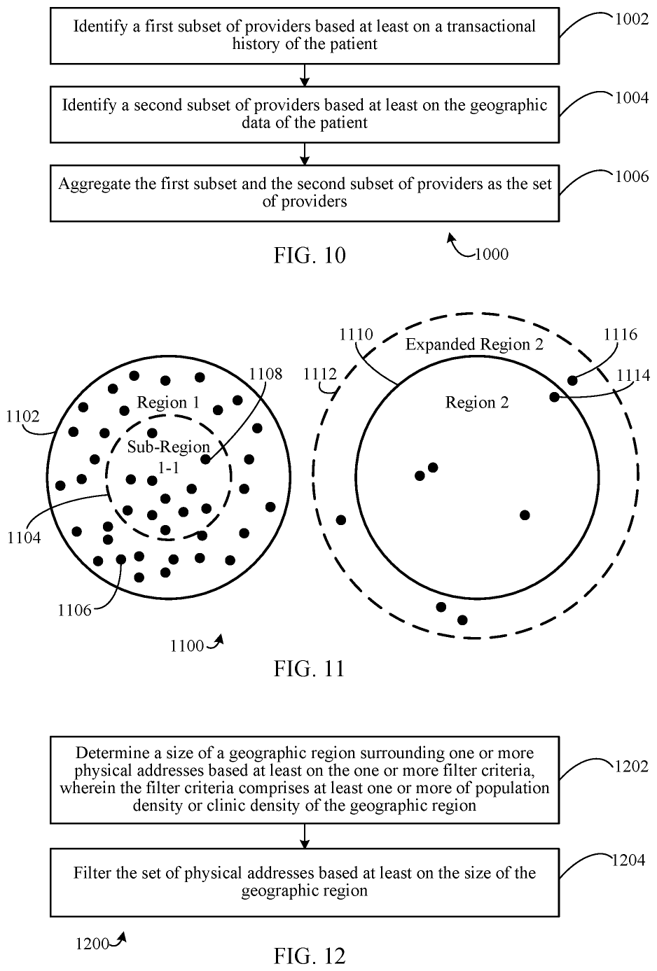

Turning now to FIG. 10, a flowchart 1000 for efficient and intelligent source reductions for querying multiple sources over an external network is shown, according to an example embodiment. Further structural and operational examples will be apparent to persons skilled in the relevant art(s) based on the following descriptions. Flowchart 1000 may be an embodiment of flowchart 400 of FIG. 4, and is described as follows with respect to FIGS. 4-5 and 8-9. In embodiments, one or more steps of flowchart 1000 may not be performed.

Flowchart 1000 starts at step 1002, which may be performed as a subset of step 404 of flowchart 400. In step 1002, a first subset of providers is identified based at least on a transactional history of the patient. For example, a first one of provider subset identifiers 506 of provider identifier 502 in FIG. 5 identifies a first subset of providers based at least on one or more of a transactional history associated with history request 518 and/or a transactional history associated with patient information 514. In a non-limiting example, history request 518 includes data indicating a transactional history between the patient and a first provider associated with first physical address of a first provider 810 of map 800 in FIG. 8, “Provider 1”, and patient information 514 includes data indicating a transactional history between the patient and a second provider associated with first physical address of a second provider 812, “Provider 2”. In the context of this non-limiting example, the first one of provider subset identifiers 506 identifies a first subset of providers including at least Provider 1 and Provider 2.

Step 1004 may be performed as a subset of step 404 of flowchart 400. In step 1004, a second subset of providers is identified based at least on geographic data of the patient. For example, a second one of provider subset identifiers 506 of provider identifier 502 in FIG. 5 identifies a second subset of providers based at least on the geographic data of the patient determined by request information determiner 504 in step 902 of FIG. 9. In a continuation of the non-limiting example described with respect to step 1002 above, the geographic data of the patient determined by request information determiner 504 in step 902 includes third region 806 of FIG. 8. In the context of this non-limiting example, the second one of provider subset identifiers 506 identifies a second subset of providers including at least Provider 1 and a third provider associated with first physical address of a third provider 820, “Provider 3”.

Step 1006 may be performed as a subset of step 404 of flowchart 400, subsequent to steps 1002 and 1004. In step 1006, the first subset and the second subset of providers are aggregated as the set of providers. For example, provider aggregator 508 of provider identifier 502 in FIG. 5 aggregates the first subset of providers identified in step 1002 by the first one of provider subset identifiers 506 and the second subset of providers identified in step 1004 by the second one of provider subset identifiers 506 as the set of providers. In a continuation of the non-limiting example described with respect to steps 1002 and 1004 above, provider aggregator 508 aggregates the first subset and the second subset of providers as a set of providers including at least Provider 1, Provider 2, and Provider 3.

In context of the non-limiting example described above with respect to the steps of flowchart 1000, step 406 of flowchart 400 in FIG. 4 may generate physical addresses associated with Provider 1, Provider 2, and Provider 3 identified in flowchart 1000. In this context, address generator 510 of provider identifier 502 identifies at least physical addresses 810-822 of map 800 in FIG. 8. In embodiments, geographic locating functions may be performed to identify providers and physical addresses the patient may have interacted with.

In the context of the method described in flowchart 1000, systems may use transactional data identifying a transactional history of a patient and geographic data of the patient to identify providers and physical addresses that may be candidates from which the history request can be satisfied. Transactional data may include, but is not limited to, medication prescriptions, past diagnosis codes, medical records, clinic visits, prescribing providers, prescription pickup locations, insurance information, and/or the like. Geographic data may include, but is not limited to, the patient's home address, the patient's work address, locations the patient has traveled to (e.g., recent vacations, work-related travel, addresses of relatives, etc.), past address information (e.g., former employer's address, past residential addresses), address type (e.g., single family home, multi-family home, apartment complex, dorm, etc.), addresses of identified providers (e.g., doctor's offices, clinics, hospitals, etc.) and/or the like. In a non-limiting example, transactional data is used to identify a primary care provider (e.g., Provider 1) whom provides primary care for the patient at a primary care office (e.g., first physical address of the first provider 810), and geographic data is used to identify physical addresses associated with the primary care provider (e.g., second physical address of the first provider 814, and third physical address of the first provider 818 as described with respect to FIG. 8), other providers with physical addresses in a region surrounding the primary care office (e.g., first physical address of a second provider 812 as described with respect to FIG. 8), and physical addresses of other providers in a region surrounding the patient's home address. That is, in some embodiments, geographic locating functions may be performed to identify providers and physical addresses the patient may have interacted with.

FIG. 11 shows a diagram of regional density 1100 (“density diagram 1100” herein) for understanding locating and filtering techniques described herein. Density diagram 1100 as exemplarily illustrated and described may be used to understand functions of a record locating and exchange system for efficient and intelligent source reductions for querying multiple sources over an external network, such as system 300 of FIG. 3.

Density diagram 1100 includes a first region 1102 and a second region 1110, corresponding to respective geographic regions covering respective particular areas. In the illustrated example of density diagram 1100, physical addresses of respective providers are represented as nodes within their respective regions. For example, first region 1102 includes many physical addresses, including a first physical address 1106 and a second physical address 1108. First region 1102 includes sub-region 1104, which includes a subset of physical addresses associated with first region 1102. In the illustrated example of density diagram 1100, second physical address 1108 is included in sub-region 1104 and first physical address 1106 is excluded from sub-region 1104. Second region 1110 includes relatively fewer physical addresses than first region 1102, including third physical address 1114. Density diagram 1100 includes an expanded region 1112, which may include additional physical addresses of respective providers, including fourth physical address 1116.

As illustrated, density diagram 1100, first region 1102, second region 1110, sub-region 1104, expanded region 1112, and respective physical addresses in FIG. 1100 are not drawn to scale. For example, first region 1102 and second region 1110 are shown as circles of equal size, however each region may be shaped and/or sized differently. In some embodiments respective regions may have a default size and/or shape (e.g., a set radius surrounding a point, a set acreage, a predetermined geographic boundary, etc.). In FIG. 1100, sub-region 1104 is shown as centered with respect to first region 1102, however sub-regions may be positioned, shaped, and/or sized in a variety of ways with respect to their respective region. For example, a sub-region may be shaped with respect to a city-block. Furthermore, a region may be subdivided into multiple sub-regions. For example, a region corresponding to a city may be subdivided into multiple sub-regions corresponding to one or more city blocks and/or zip codes. In FIG. 1100, expanded region 1112 is shown expanding the area of second region 1110 equidistant from its edge, however expanded regions may be positioned, shaped, and/or sized in a variety of ways with respect to their respective region. For example, an expanded region may expand a region representing a city to encompass an adjacent suburban area. FIG. 1100, as illustrated, shows physical addresses as nodes (e.g., first physical address 1106, second physical address 1108, third physical address 1114, and fourth physical address 1116). It is contemplated herein that any region, sub-region, and/or expanded region may contain more or fewer physical addresses. For example, a region representing a densely populated city may contain hundreds, thousands, or more physical addresses of respective providers, however, a rural town may contain ten or fewer physical addresses of respective providers. In some cases, any two or more physical addresses within a region, sub-region, and/or expanded region may be associated with one or more similar providers. For example, a provider may be a doctor with a primary care office in the same region as a hospital they hold office hours at.

Turning now to FIG. 12, a flowchart 1200 for efficient and intelligent source reductions for querying multiple sources over an external network is shown, according to an example embodiment. Further structural and operational examples will be apparent to persons skilled in the relevant art(s) based on the following descriptions. Flowchart 1200 may be an embodiment of flowchart 400 of FIG. 4, and is described as follows with respect to FIGS. 4, 6, and 9-11. In embodiments, one or more steps of flowchart 1200 may not be performed.

Flowchart 1200 starts at step 1202, which may be performed as a subset of either step 408 or step 410 of flowchart 400. In step 1202, a size of a geographic region surrounding one or more physical addresses is determined based at least on one or more filter criteria. In this context, the filter criteria may comprise at least one or more of population density or clinic density of the geographic region. For example, the geographic region of step 1202 may be determined according to embodiments described above, including but not limited to as the steps described with respect to FIG. 9 and/or FIG. 10. In this context, the size of the geographic region may be determined by filtration determiner 604 and/or filter engine 606 of address filter 602 in FIG. 6 based at least on one or more of the geographic region, set of physical addresses 618, and/or filter criteria 614. Information pertaining to clinic density may include or be measured by the number of clinics within the geographic region, the average distance between clinics within the geographic region, and/or the like. In this context, clinics may correspond to all or a subset of providers and/or physical addresses associated with providers.

Step 1204 may be performed as a subset of step 410 of flowchart 400, subsequent to step 1202. In step 1204, the set of physical addresses are filtered based at least on the size of the geographic region. For example, filter engine 606 of address filter 602 in FIG. 6 filters set of physical addresses 618 based at least on the size of the geographic region determined in step 1202 to generate filtered set of physical addresses 620. In this context, embodiments described herein may include methods for dynamically adjusting geographic search areas to efficiently and intelligently ensure that the reduced, filtered set are determined as candidates from which the request can be satisfied, but still without querying each computing system at each determined physical address.

In a first non-limiting example of flowchart 1200, set of physical addresses 618 of FIG. 6 includes at least first physical address 1106 and second physical address 1108 of FIG. 11. In this example, first region 1102 is provided as a geographic region surrounding one or more physical addresses, including at least first physical address 1106 and second physical address 1108. In this context, filtration determiner 604 and/or filter engine 606 of address filter 602 in FIG. 6 determine a size of the geographic region, as described with respect to step 1202, by selecting an area corresponding to sub-region 1104 based at least on the clinic density of first region 1102 being at or above a threshold, or the like. Filter engine 606 filters set of physical addresses 618 based at least on the area corresponding to sub-region 1104. In this example, filter engine 606 removes at least first physical address 1106 from filtered set of physical addresses 620.

In a second non-limiting example of flowchart 1200, set of physical addresses 618 of FIG. 6 includes at least third physical address 1114 and fourth physical address 1116 of FIG. 11. In this example, second region 1110 is provided as a geographic region surrounding one or more physical addresses, including at least third physical address 1114. In this context, filtration determiner 604 and/or filter engine 606 of address filter 602 in FIG. 6 determine a size of the geographic region, as described with respect to step 1202, by selecting an area corresponding to expanded region 1112 based at least on the clinic density of second region 1110 being at or below a threshold, and/or the like. Filter engine 606 filters set of physical addresses 618 based at least on the area corresponding to expanded region 1112. In this example, filter engine 606 includes at least fourth physical address 1116 as part of filtered set of physical addresses 620 where it would have otherwise been filtered from filtered set of physical addresses 620. It is contemplated that the use of expanded regions, such as expanded region 1112 as described with respect to this second non-limiting example of flowchart 1200, may be used to identify a second subset of providers as described with respect to step 1004 in flowchart 1000 of FIG. 10.

Flowchart 1200 provides further understanding of filtering a set of physical addresses according to filter criteria based at least on one or more of population density and/or clinic density of the geographic region. Several examples of embodiments performing functions of flowchart 1200 have been described above, however it is contemplated herein that steps of flowchart 1200 may be performed in a variety of ways, as described herein or as otherwise would be understood by persons of skill in the relevant art(s) having the benefit of this disclosure. For instance, geographic regions may or may not be increased and/or reduced in size, changed in shape, and/or otherwise determined based on filter criteria. Additionally, filter criteria beyond clinic density and/or population density may be used to further filter set of physical addresses 618 and generate filtered set of physical addresses 620.

IV. Example Machine Learning System Embodiments

As described above, systems may be configured in various ways to perform their described functions. For instance, systems may be configured to generate a patient profile for a patient where the profile is associated with information including, but not limited to, patient identification, geographical information, family relationships, prescription pickup locations, patient demographics, provider interactions, medication history, prior diagnosis codes, insurance information, transactional data, and/or the like, according to examples described herein.

Training of ML models based on information associated with the patient profile may be performed according to the described techniques and embodiments. For example, a patient profile may be modeled using ML techniques based on information associated with the patient. The patient profile may be stored within a database comprising multiple patient profiles. A trained matching model may be trained on prior history requests and/or transactional data of the patient. Parameters of the model may be directed to various aspects of patient information (e.g., patient identification, transactional data, patient demographics, provider interactions, medication history, prior diagnosis codes, insurance information, geographical information, family relationships, prescription pickup locations, etc.). Parameters may have weights assigned to them based on the likelihood of a match.

The trained matching model may assign a score or index to each patient profile stored within a database (“stored patient profiles”), or otherwise evaluated by the model, indicating a likelihood of matching the patient profile associated with a received history request (“received patient profile”). For example, stored patient profiles with many matching criteria with the received patient profile may have a higher score than stored patient profiles with little or no matching criteria with the received patient profile. In this context, the trained matching model may have a threshold value that scores/indices are required to meet or exceed to determine a positive match (while other comparisons to thresholds are also contemplated herein). This threshold value may be predetermined, adjustable by a model or component of the system, or set external to the system. It is contemplated herein that matching scores/indices within a predetermined amount of meeting/exceeding such a threshold may be identified for further refinement of the trained matching model.

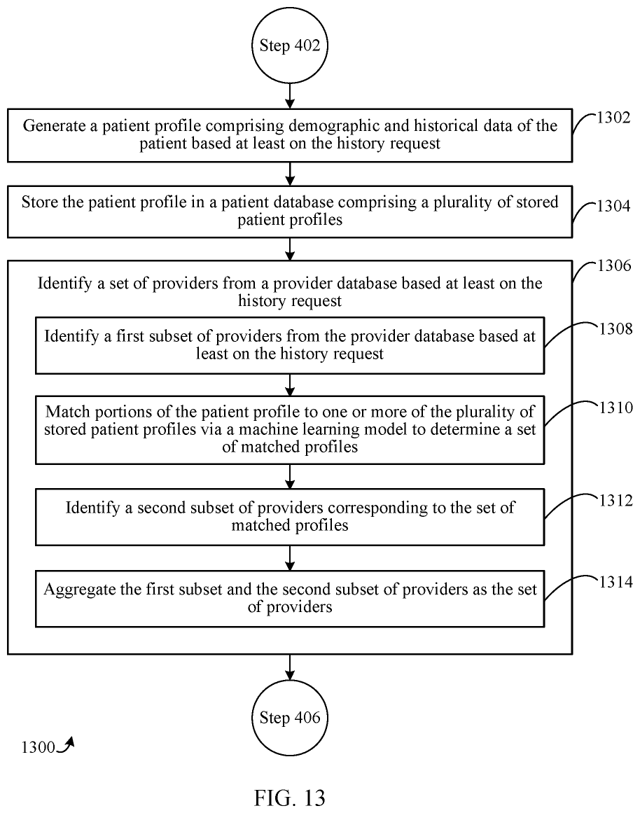

Turning now to FIG. 13, a flowchart 1300 for efficient and intelligent source reductions for querying multiple sources over an external network is shown, according to an example embodiment. Further structural and operational examples will be apparent to persons skilled in the relevant art(s) based on the following descriptions. Flowchart 1300 may be an embodiment of flowchart 400, and is described as follows with respect to FIGS. 2-5, 8, and 10. In embodiments, one or more steps of flowchart 1300 may not be performed.

Flowchart 1300 starts at step 1302, which may be performed subsequent to step 402 of flowchart 400 in FIG. 4. In step 1302, a patient profile comprising demographic and historical data of a patient is generated based at least on the history request. For example, request receiver 308 of system 300 in FIG. 3 receives a history request (which may correspond to history request 518 of FIG. 5) from first requestor system 206 and/or second requestor system 208 via network 210 of FIG. 2. In this context, request receiver 308 stores information pertaining to the patient associated with the history request as a portion of patient information 310. One of ML model(s) 320 accesses patient information 310 to generate the patient profile comprising at least demographic and historical data of the patient.

In step 1304, the patient profile is stored in a patient database comprising a plurality of stored patient profiles. For example, the patient profile generated by the one of ML model(s) 320 may be stored in a database, such as DB(s) 326 of system 300 in FIG. 3.

Step 1306 may be a further embodiment of step 404 of flowchart 400 in FIG. 4. In step 1306, a set of providers are identified from a provider database based at least on the history request. For example, provider identifier 502 of FIG. 5 identifies a set of providers from provider directory 516 based at least on information in history request 518. Step 1306 may include one or more subsets of steps as described in the following examples, as described with respect to above embodiments, and/or as otherwise would be understood by persons of skill in the relevant art(s) having benefits of this disclosure.

Step 1308 may be a subset of step 1306, and/or may be a further embodiment of step 404 of FIG. 4 and/or step 1002 of FIG. 10. In step 1308, a first subset of providers is identified from the provider database based at least on the history request. For example, a first one of provider subset identifiers 506 of provider identifier 502 in FIG. 5 identifies a first subset of providers from provider directory 516 based at least on history request 518.

Step 1310 may be a subset of step 1306. In step 1310, portions of the patient profile are matched to one or more of the plurality of stored patient profiles via a machine learning model (ML model) to determine a set of matched profiles. For example, a trained matching model of ML model(s) 320 of system 300 in FIG. 3 matches portions of the patient profile associated with history request 518 to one or more of the plurality of stored patient profiles to determine a set of matched profiles. In embodiments, the matched profiles may be determined based at least on respective scores and/or indices exceeding or meeting a threshold value. In a non-limiting example, the patient profile associated with history request 518 may indicate that the patient receives primary care from a first provider associated with the first physical address of the first provider 810 of map 800 in FIG. 8, “Provider 1”, resides in first region 802, “Region 1”, works in second region 804, “Region 2”, has been given a previous diagnosis, “Diagnosis D”, and has been prescribed a medication, “Medication M”. The trained matching model of ML model(s) 320 matches the patient profile with one of the plurality of stored patient profiles indicating that the matched patient receives primary care from Provider 1, resides in Region 1, has been given Diagnosis D, and has been prescribed Medication M.

Step 1312 may be a subset of step 1306. In step 1312, a second subset of providers is identified from the provider database corresponding to the set of matched profiles. For example, a second one of provider subset identifiers 506 of provider identifier 502 in FIG. 5 identifies a second subset of providers from provider directory 516 corresponding to the set of matched profiles determined in step 1310. In the context of the non-limiting example described with respect to step 1310, the matched patient has also visited a second provider associated with the first physical address of the second provider 812, “Provider 2”, and a third provider associated with the first physical address of the third provider 820, “Provider 3”, of map 800 in FIG. 8. In this non-limiting example, the second one of provider subset identifiers 506 identifies the second subset of providers including at least Provider 2 and Provider 3.

Step 1314 may be a subset of step 1306, and/or may be a further embodiment of step 1006 of FIG. 10. In step 1314, the first subset and the second subset of providers are aggregated as the set of providers. For example, provider aggregator 508 of provider identifier 502 in FIG. 5 aggregates the first subset of providers identified in step 1308 and the second subset of providers identified in step 1312 as the set of providers. In the context of the non-limiting example described with respect to step 1310 and step 1312, a first one of provider subset identifiers 506 may identify at least Provider 1 as the first subset of providers. In this context, provider aggregator 508 aggregates the first subset and the second subset of providers as a set of providers including at least Provider 1, Provider 2, and Provider 3.

In the context of the non-limiting example described above with respect to step 1310 and step 1312, step 406 of flowchart 400 may generate physical addresses associated with Provider 1, Provider 2, and Provider 3 identified in flowchart 1300. In this context, address generator 510 of provider identifier 502 identifies at least physical addresses 810-822 of map 800 in FIG. 8. In embodiments, ML models may be used to identify providers and physical addresses the patient may have interacted with.

In some embodiments, ML models described with respect to FIG. 13 and the examples above may be used with the steps described in flowchart 700 of FIG. 7. For example, likelihood scores calculated in step 702 of flowchart 700 may be based at least on scores and/or indices of matched profiles identified by ML models in step 1310. In this context, a higher score and/or index may contribute to a higher likelihood score.

It is also contemplated herein that while flowchart 1300 of FIG. 13 is described as being an embodiment of flowchart 400 of FIG. 4, embodiments herein are not so limited, and one or more steps of flowchart 1300 may be performed at different times and/or in different contexts associated with other embodiments herein.

V. Further Example Embodiments and Advantages

As noted above, systems and devices, including record locating and exchange systems, may be configured in various ways for efficient and intelligent source reductions for querying multiple sources over an external network. Historic records have been described herein as medical records of a patient, however it is also contemplated herein that historic records may pertain to geographic records of the patient, demographic records of the patient, transaction records of the patient, or the like. Data resources have been described as data stored in system memory, internal databases, and/or external databases. Filter criteria has been described in various forms of demographic criteria, geographic criteria, medical criteria, and/or the like, however, it is also contemplated herein that other filter criteria may be used, including, but not limited to, economic factors (e.g. income, credit, etc.), home address type (e.g., apartment, single-family home, multi-family home, dorm, etc.), travel history and/or the like. The systems and devices described herein are utilized to determine medical history records for a patient.

The described techniques and embodiments provide a system with the ability to automatically identify providers and/or physical addresses that may have medical records pertaining to a patient. For example, a set of providers and/or physical addresses may be identified based at least on one or more of information in a history request, stored information pertaining to the patient, stored information pertaining to providers, geographic information, and/or machine learning utilizing stored profiles of multiple patients.

The systems and devices herein may identify providers and/or physical addresses a patient interacts with that may be missed due to factors such as, but not limited to, a patient's incomplete or inaccurate memory, a restricted geographic profile, and/or the like. Typically, a requestor of a history request wants all or as many as possible of historic records associated with a patient. Techniques and embodiments described herein are utilized to determine as many providers to query for historic records to determine a complete or near-complete historic record for a patient.

The systems and devices herein may identify hundreds or thousands of potential providers and/or physical addresses that may be queried for medical records pertaining to a patient. Due to system and/or network constraints it may be infeasible to query every identified potential provider and/or physical address.

In anon-limiting example, over 120 million history requests may be made in a single day in a network of computing systems including a host system with a record locating and exchange system and tens of thousands of computing systems associated with respective requestors and/or providers. Without filtering, each computing system associated with a provider would be queried for each history request in order to determine a complete history request for a respective patient. Performing a query on each computing system for each history request may be difficult or infeasible due to constraints of the host system, the network, and/or the computing systems of the respective requestors and/or providers.

The described techniques and embodiments provide for a specific arrangement of components for filtering the set of physical addresses and/or providers to a manageable amount. The techniques and embodiments described herein filter the set of physical addresses and/or providers according to filter criteria such as, but not limited to, likelihood scores, weight values, geographic information, demographic information, machine learning profiles, and/or the like. That is, the set of physical addresses and/or providers are filtered to a filtered set that are likely to have historic records pertaining to the patient. In this way, the techniques and embodiments described herein may reduce strain on the host system, the network, and/or the computing systems of the respective requestors and/or providers. That is, efficient and intelligent source reductions for querying multiple sources over an external network includes reducing candidate physical addresses, e.g., via filtering, to generate a manageable set of physical addresses to be queried, which prevents system crashes, reduces memory footprint, reduces processing cycles requires, and reduces network bandwidth, while at the same time, ensuring that physical addresses in the reduced, filtered set are determined as candidates from which the request can be satisfied.

The described techniques and embodiments may be utilized to query computing systems corresponding to the reduced, filtered set of physical addresses. Queries performed over a network, which may be an external network with respect to the querying system, and which may include querying hundreds, thousands, or more computing systems for the filtered set of physical addresses in different scenarios and geographic regions, again ensuring that physical addresses in the reduced, filtered set are determined as candidates from which the request can be satisfied, but still without querying each computing system at each determined physical address.

The described techniques and embodiments may be utilized as or in any computing device or distributed computing system. The described techniques and embodiments provide value and efficiency benefits for large, and still increasing, networks of hosts, health care providers, and trading partners that desire to exchange historic records.

Machine learning models, e.g., the matching ML model, as described herein, may be modified via training on parameters such as history requests, patient information, provider databases, and/or the like. The matching ML model may be retrained on a set routine basis and/or based at least on one or more event. Types of events include, but are not limited to, a critical mass of changes in patient information, a change in residency, a change in insurance, a set time when new insurance plans are activated (e.g., the first of January), a change in top medications prescribed a rolling time period, a cyclical occurrence (e.g., “Flu Season”), a threshold change in the provider director, a pandemic, placement or release of travel restrictions, a natural disaster, and/or the like.

Moreover, according to the described embodiments and techniques, any components of record locating and exchange systems and their functions may be caused to be activated for operation/performance thereof based on other operations, functions, actions, and/or the like, including initialization, completion, and/or performance of the, functions, actions, and/or the like.

In some example embodiments, one or more of the operations of the flowcharts, flow diagrams, and/or execution flows described herein may not be performed. Moreover, operations in addition to or in lieu of the operations of the flowcharts, flow diagrams, and/or execution flows described herein may be performed. Further, in some example embodiments, one or more of the operations of the flowcharts, flow diagrams, and/or execution flows described herein may be performed out of order, in an alternate sequence, or partially (or completely) concurrently with each other or with other operations.

The further example embodiments and advantages described in this Section may be applicable to any embodiments disclosed in this Section or in any other Section of this disclosure.

Embodiments and techniques, including methods, described herein may be performed in various ways such as, but not limited to, being implemented by hardware, or hardware combined with one or both of software and firmware.

VI. Example Processing Device Implementations

Record locating and exchange system embodiments described herein, such as record locating and exchange system 104 of FIG. 1, record locating and exchange system 204 of FIG. 2, and/or record locating and exchange system 300 of FIG. 3, along with any respective components/subcomponents and/or further embodiments thereof, and/or any flowcharts, flow diagrams, execution flows, further systems, sub-systems, and/or components, including other network-connected devices, disclosed herein may be implemented in hardware (e.g., hardware logic/electrical circuitry), or any combination of hardware with one or both of software (computer program code or instructions configured to be executed in one or more processors or processing devices) and firmware. In embodiments with respect to the example computer implementations in this Section, main memory, memory cards and memory sticks, memory devices, and/or the like may include and or implement the described techniques and embodiments.

The embodiments described herein, including devices, systems, methods/processes, and/or apparatuses, may be implemented in or using processing devices, communication systems, servers, and/or, computers, such as a processing device 1400 shown in FIG. 14. It should be noted that processing device 1400 may represent mobile devices, communication devices/systems, entertainment systems/devices, processing devices, and/or traditional computers in one or more embodiments. For example, a system as described herein, and any of the sub-systems and/or components respectively contained therein and/or associated therewith, along with further embodiments thereof, may be implemented in or using one or more processing devices 1400 and/or similar computing devices.

Processing device 1400 can be any commercially available and well known communication device, processing device, and/or computer capable of performing the functions described herein, such as devices/computers available from International Business Machines®, Apple®, Sun®, HP®, Dell®, Cray®, Samsung®, Nokia®, etc. Processing device 1400 may be any type of computer, including a desktop computer, a server, etc., and may be a computing device or system within another device or system.

Processing device 1400 includes one or more processors (also called central processing units, or CPUs), such as a processor 1406. Processor 1406 is connected to a communication infrastructure 1402, such as a communication bus. In some embodiments, processor 1406 can simultaneously operate multiple computing threads, and in some embodiments, processor 1406 may comprise one or more processors.

Processing device 1400 also includes a primary or main memory 1408, such as random access memory (RAM). Main memory 1408 has stored therein control logic 1424 (computer software), and data.

Processing device 1400 also includes one or more secondary storage devices 1410. Secondary storage devices 1410 include, for example, a hard disk drive 1412 and/or a removable storage device or drive 1414, as well as other types of storage devices, such as memory cards and memory sticks. For instance, processing device 1400 may include an industry standard interface, such a universal serial bus (USB) interface for interfacing with devices such as a memory stick. Removable storage drive 1414 represents a floppy disk drive, a magnetic tape drive, a compact disk drive, an optical storage device, tape backup, etc.

Removable storage drive 1414 interacts with a removable storage unit 1416. Removable storage unit 1416 includes a computer useable or readable storage medium 1418 having stored therein computer software 1426 (control logic) and/or data. Removable storage unit 1416 represents a floppy disk, magnetic tape, compact disk, DVD, optical storage disk, or any other computer data storage device. Removable storage drive 1414 reads from and/or writes to removable storage unit 1416 in a well-known manner.

Processing device 1400 also includes input/output/display devices 1404, such as touchscreens, LED and LCD displays, monitors, keyboards, pointing devices, etc.

Processing device 1400 further includes a communication or network interface 1420. Communication interface 1420 enables processing device 1400 to communicate with remote devices. For example, communication interface 1420 allows processing device 1400 to communicate over communication networks or mediums 1422 (representing a form of a computer useable or readable medium), such as LANs, WANs, the Internet, etc. Network interface 1420 may interface with remote sites or networks via wired or wireless connections.

Control logic 1428 may be transmitted to and from processing device 1400 via the communication medium 1422.

Any apparatus or manufacture comprising a computer useable or readable medium having control logic (software) stored therein is referred to herein as a computer program product or program storage device. This includes, but is not limited to, processing device 1400, main memory 1408, secondary storage devices 1410, and removable storage unit 1416. Such computer program products, having control logic stored therein that, when executed by one or more data processing devices, cause such data processing devices to operate as described herein, represent embodiments.

Techniques, including methods, and embodiments described herein may be implemented by hardware (digital and/or analog) or a combination of hardware with one or both of software and/or firmware. Techniques described herein may be implemented by one or more components. Embodiments may comprise computer program products comprising logic (e.g., in the form of program code or software as well as firmware) stored on any computer useable medium, which may be integrated in or separate from other components. Such program code, when executed by one or more processor circuits, causes a device to operate as described herein. Devices in which embodiments may be implemented may include storage, such as storage drives, memory devices, and further types of physical hardware computer-readable storage media. Examples of such computer-readable storage media include, a hard disk, a removable magnetic disk, a removable optical disk, flash memory cards, digital video disks, random access memories (RAMs), read only memories (ROM), and other types of physical hardware storage media. In greater detail, examples of such computer-readable storage media include, but are not limited to, a hard disk associated with a hard disk drive, a removable magnetic disk, a removable optical disk (e.g., CDROMs, DVDs, etc.), zip disks, tapes, magnetic storage devices, MEMS (micro-electromechanical systems) storage, nanotechnology-based storage devices, flash memory cards, digital video discs, RAM devices, ROM devices, and further types of physical hardware storage media. Such computer-readable storage media may, for example, store computer program logic, e.g., program modules, comprising computer executable instructions that, when executed by one or more processor circuits, provide and/or maintain one or more aspects of functionality described herein with reference to the figures, as well as any and all components, capabilities, and functions therein and/or further embodiments described herein.

Such computer-readable media and/or computer-readable storage media (e.g., a computer-readable storage medium, or other derivative thereof) are distinguished from and non-overlapping with communication media and propagating signals (do not include communication media and propagating signals). Communication media embodies computer-readable instructions, data structures, program modules or other data in a modulated data signal such as a carrier wave. The term “modulated data signal” means a signal that has one or more of its characteristics set or changed in such a manner as to encode information in the signal. By way of example, and not limitation, communication media includes wireless media such as acoustic, RF, infrared and other wireless media, as well as wired media. Embodiments are also directed to such communication media that are separate and non-overlapping with embodiments directed to computer-readable storage media.

The techniques and embodiments described herein may be implemented as, or in, various types of circuits, devices, apparatuses, and systems. For instance, embodiments may be included, without limitation, in processing devices (e.g., illustrated in FIG. 14) such as computers and servers, as well as communication systems such as switches, routers, gateways, and/or the like, communication devices such as smart phones, home electronics, gaming consoles, entertainment devices/systems, etc. A device, as defined herein, is a machine or manufacture as defined by 35 U.S.C. § 101. That is, as used herein, the term “device” refers to a machine or other tangible, manufactured object and excludes software and signals. Devices may include digital circuits, analog circuits, or a combination thereof. Devices may include one or more processor circuits (e.g., central processing units (CPUs), processor 1406 of FIG. 14), microprocessors, digital signal processors (DSPs), and further types of physical hardware processor circuits) and/or may be implemented with any semiconductor technology in a semiconductor material, including one or more of a Bipolar Junction Transistor (BJT), a heterojunction bipolar transistor (HBT), a metal oxide field effect transistor (MOSFET) device, a metal semiconductor field effect transistor (MESFET) or other transconductor or transistor technology device. Such devices may use the same or alternative configurations other than the configuration illustrated in embodiments presented herein.

VII. Conclusion

While various embodiments have been described above, it should be understood that they have been presented by way of example only, and not limitation. It will be apparent to persons skilled in the relevant art that various changes in form and detail can be made therein without departing from the spirit and scope of the embodiments. Thus, the breadth and scope of the embodiments should not be limited by any of the above-described exemplary embodiments, but should be defined only in accordance with the following claims and their equivalents.

What is claimed is:

1. A computer-implemented method performed by a host system, comprising:
receiving, from a requestor system and via a network external to the host system, a history request for a patient;
identifying a set of providers from a provider database based at least on the history request;
generating a set of physical addresses based at least on the set of providers, the set of physical addresses comprising geographic data for each provider associated with the set of providers;
determining one or more filter criteria based at least on one or more of the history request or the set of providers;
filtering the set of physical addresses according to the filter criteria to generate a filtered set of physical addresses that comprises at least one less physical address than the set of physical addresses; and
querying computing systems, of respective providers, that respectively correspond to ones of the filtered set of physical addresses, via the network, to generate a query result that includes history information for the patient, the query result being representative of querying each computing system associated with the set of physical addresses.

2. The computer-implemented method of claim 1, further comprising:
determining geographic data of the patient based at least on the history request; and
wherein said identifying the set of providers from the provider database comprises:
identifying a first subset of providers based at least on a transactional history of the patient;
identifying a second subset of providers based at least on the geographic data of the patient; and
aggregating the first subset and the second subset of providers as the set of providers.

3. The computer-implemented method of claim 2, wherein filtering the set of physical addresses comprises:
determining a size of a geographic region surrounding one or more physical addresses based at least on the one or more filter criteria, wherein the filter criteria comprises at least one or more of population density or clinic density of the geographic region; and
filtering the set of physical addresses based at least on the size of the geographic region.

4. The computer-implemented method of claim 1, further comprising:
generating a patient profile comprising demographic and historical data of the patient based at least on the history request;
storing the patient profile in a patient database comprising a plurality of stored patient profiles; and
wherein said identifying the set of providers from the provider database comprises:
identifying a first subset of providers from the provider database based at least on the history request;
matching portions of the patient profile to one or more of the plurality of stored patient profiles via a machine learning model to determine a set of matched profiles;
identifying a second subset of providers corresponding to the set of matched profiles; and
aggregating the first subset and the second subset of providers as the set of providers.

5. The computer-implemented method of claim 1, wherein filtering the set of physical address comprises:
calculating likelihood scores for each physical address within the set of physical addresses, wherein the likelihood scores are calculated based on a likelihood that the patient interacted with a respective provider associated with a respective physical address; and
filtering the set of physical addresses based at least on respective likelihood scores.

6. The computer-implemented method of claim 5, wherein the likelihood that the patient interacted with the respective provider is based at least on an activity of the patient and a last recorded activity of the respective provider.

7. The computer-implemented method of claim 1, wherein the one or more filter criteria comprises one or more of:
a clinic density of a geographic region;
a population density of a geographic region;
a practice area;
provider activity; or
vendor responsiveness.

8. A host system comprising:
a memory that stores program code; and
a processing system, comprising one or more processors, configured to receive the program code from the memory and, in response to at least receiving the program code, to:
receive, from a requestor system and via a network external to the host system, a history request for a patient;
identify a set of providers from a provider database based at least on the history request;
generate a set of physical addresses based at least on the set of providers, the list of physical addresses comprising geographic data for each provider associated with the set of providers;
determine one or more filter criteria based at least on one or more of the history request or the set of providers;
filter the set of physical addresses according to the filter criteria to generate a filtered set of addresses that comprises at least one less physical address than the set of physical addresses; and
query computing systems, of respective providers, that respectively correspond to ones of the filtered set of physical addresses, via the network, to generate a query result that includes history information for the patient, the query result being representative of querying each computing system associated with the set of physical addresses.

9. The host system of claim 8, wherein the processing system is further configured to:
determine geographic data of the patient based at least on the history request; and
wherein said identify the set of providers from the provider database includes:
to identify a first subset of providers based at least on a transactional history of the patient;
to identify a second subset of providers based at least on the geographic data of the patient; and
to aggregate the first subset and the second subset of providers as the set of providers.

10. The host system of claim 9, wherein said filter the set of physical addresses comprises:
to determine a size of a geographic region surrounding one or more addresses based at least on the one or more filter criteria, wherein the filter criteria comprises at least one or more of population density or clinic density of the geographic region; and
to filter the set of physical addresses based at least on the size of the geographic region.

11. The host system of claim 8, wherein the processing system is further configured to:
generate a patient profile comprising demographic and historical data of the patient based at least on the history request;
store the patient profile in a patient database comprising a plurality of stored patient profiles; and
wherein said identify the set of providers from the provider database includes:
to identify a first subset of providers from the provider database corresponding to the history request;
to match portions of the patient profile to one or more of the plurality of stored patient profiles via a machine learning model to determine a set of matched profiles;
to identify a second subset of providers corresponding to the set of matched profiles; and
to aggregate the first subset and the second subset of providers as the set of providers.

12. The host system of claim 8, wherein said filter the set of physical addresses comprises:
to calculate likelihood scores for each physical address within the set of physical addresses, wherein the likelihood scores are calculated based on a likelihood that the patient interacted with a respective provider associated with a respective physical address; and
to filter the set of physical addresses based at least on respective likelihood scores.

13. The host system of claim 12, wherein the likelihood that the patient interacted with the respective provider is based at least on an activity of the patient and a last recorded activity of the respective provider.

14. The host system of claim 8, wherein the one or more filter criteria comprises one or more of:
a clinic density of a geographic region;
a population density of a geographic region;
a practice area;
provider activity; or
vendor responsiveness.

15. A computer-readable storage medium having programming instructions encoded thereon that are executable by one or more processors to perform a method, the method comprising:
receiving, from a requestor system and via a network external to the computer-readable storage medium, a history request for a patient;
identifying a set of providers from a provider database based at least on the history request;
generating a set of physical addresses based at least on the set of providers, the set of physical addresses comprising geographic data for each provider associated with the set of providers;
determining one or more filter criteria based at least on one or more of the history request or the list of providers;
filtering the set of physical addresses according to the filter criteria to generate a filtered set of physical addresses that comprises at least one less physical address than the set of physical addresses; and
querying computing systems, of respective providers, that respectively correspond to ones of the filtered set of physical addresses, via the network, to generate a query result that includes history information for the patient, the query result being representative of querying each computing system associated with the set of physical addresses.

16. The computer-readable storage medium of claim 15, wherein the method further comprises:
determining geographic data of the patient based at least on the history request; and
wherein said identifying the list of providers from the provider database comprises:
identifying a first subset of providers based at least on a transactional history of the patient;
identifying a second subset of providers based at least on the geographic data of the patient; and
aggregating the first subset and the second subset of providers as the set of providers.

17. The computer-readable storage medium of claim 16, wherein said filtering the set of physical addresses comprises:
determining a size of a geographic region surrounding one or more physical addresses based at least on the one or more filter criteria, wherein the filter criteria comprises at least one or more of population density or clinic density of the geographic region; and
filtering the set of physical addresses based at least on the size of the geographic region.

18. The computer-readable storage medium of claim 15, wherein the method further comprises:
generating a patient profile comprising demographic and historical data of the patient based at least on the history request;
storing the patient profile in a patient database comprising a plurality of stored patient profiles; and
wherein said identifying the set of providers comprises:
identifying a first subset of providers from the provider database corresponding to the history request;
matching portions of the patient profile to one or more of the plurality of stored patient profiles via a machine learning model to determine a set of matched profiles;
identifying a second subset of providers corresponding to the set of matched profiles; and
aggregating the first subset and the second subset of providers as the set of providers.

19. The computer-readable storage medium of claim 15, wherein said filtering the set of physical addresses comprises:
calculating likelihood scores for each physical address within the set of physical addresses, wherein the likelihood scores are calculated based on a likelihood that the patient interacted with a respective provider associated with a respective physical address; and
filtering the set of physical addresses based at least on respective likelihood scores.

20. The computer-readable storage medium of claim 19, wherein the likelihood that the patient interacted with the respective provider is based at least on an activity of the patient and a last recorded activity of the respective provider.
